(19) United States
(12) Patent Application Publication
(10) Pub. No.: US 2007/0232899 A1
(43) Pub. Date: Oct. 4, 2007

(54) Method for localizing an invasive instrument, and an invasive instrument

(75) Inventors: Ulrich Bill, Effeltrich (DE); Jan Boese, Eckental (DE); Norbert Rahn, Forchheim (DE); Bernhard Sandkamp, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/729,756

(22) Filed: Mar. 29, 2007

(30) Foreign Application Priority Data
Mar. 30, 2006 (DE) 10 2006 014 883.5

(51) Int. Cl. A61B 5/05

(52) U.S. Cl. 600/424; 600/407

(57) ABSTRACT

The invention is based on a new type of method which can be used to localize magnetizable small particles. According to the invention an invasive instrument, a catheter for example, is provided with a magnetizable marker, whereby a magnetic fluid is arranged in a fluid container. Such an invasive instrument can be localized during an invasive intervention on a patient by using a coil system which on the one hand generates an inhomogeneous basic magnetic field and on the other hand generates a superimposed temporally varying magnetic field. Previous localization methods, which are considerably more complex, can be dispensed with.

Method for localizing an invasive instrument, and an invasive instrument

The invention relates to a method for localizing an invasive instrument, and also a new type of invasive instrument or a set comprising at least two such new-type invasive instruments.

Invasive instruments are instruments which in the case of (minimally) invasive medical applications are introduced into the body of a patient. The concept of the invasive instrument includes for example catheters, guide wires or also stents (as are used in cardiology/angiography). Furthermore, this concept also includes needles, which are used for example in TIPS procedures, biopsy instruments or also gastrointestinal probes.

A common problem experienced by attending doctors with this diversity of invasive instruments consists in the precise positioning of the invasive instrument to a desired location. Basically, support with the aid of imaging devices is required in this situation.

It is thus usual, for example, to use electromagnetic localization systems (for example the CARTO system from the company Biosense Webster) for the purpose of three-dimensional realtime localization of catheters during electrophysiological procedures. Special catheters with position sensors are used in this situation. The position sensors are relatively large and limit the minimum constructional size of the instruments. The catheters equipped with a position sensor are also extremely expensive. During the invasive intervention, an electrical connection must be maintained between the tip of the catheter and the other end of the catheter.

The Localisa system from the company Medtronic permits the three-dimensional localization of catheter electrodes with the aid of electrical impedance measurements. Here too an electrical connection is required between the catheter and the outside.

A method is also known for mapping invasive instruments together with the patient in x-ray images during the intervention. This has the disadvantage that patient and doctor are subjected to ionizing radiation. In addition, the invasive instruments to be localized must exhibit a sufficiently high x-ray contrast, which is not always the case. By way of example, catheters consist mainly of plastic which can hardly be recognized in the x-ray image.

The object of the invention is to support a doctor when making an invasive intervention on a patient by setting down an improved method for localizing an invasive instrument, whereby the method should be both simple to implement and also reliable, and whereby it should in particular avoid the consequences of the aforementioned problems regarding localization associated with the prior art.

The object is achieved according to the invention by a method for locating an invasive instrument according to claim 1, by an invasive instrument according to claim 7 and also by a set comprising at least two invasive instruments according to claim 11.

The invention utilizes a new type of imaging method. This is described in the article by Bernhard Gleich and Jürgen Weizenecker, “Tomographic imaging using the nonlinear response of magnetic particles”, Nature, Vol 435/30 June 2005, pp. 214 to 217. The method is also presented in an article by Andreas Trabesinger, “Particular magnetic insights”, pp. 1173 to 1174 in the same volume of Nature.

The method according to the invention is implemented as follows: Firstly, an invasive instrument is made available which includes a magnetizable element. The invasive instrument in question can be a special instrument equipped with a magnetizable element. However, should an invasive instrument from the prior art already include a magnetizable element, then it is also quite possible to use this to implement the method according to the invention.

By using three pairs of coils for example, an inhomogeneous magnetic field which is temporally constant for the duration of one test step is now generated in an overall volume in which the invasive instrument is situated. The overall volume ideally includes the entire path from an entry point at which the invasive instrument is inserted into the patient to the target. This would ensure that the invasive instrument is in any event situated in the overall volume. The inhomogeneity of the magnetic field is now chosen as follows: The volume should be divided up into a multiplicity of subvolumes. These can in particular be larger than the magnetizable element in each case, with the result that it is necessary to distinguish whether or not the magnetizable element is located in the subvolume. Alternatively, the subvolumes can however also be chosen to be smaller.

Regardless of the size of the subvolumes, these are however defined such that a test volume can be distinguished from the other subvolumes. The inhomogeneous magnetic field is chosen such that in all the subvolumes except for one test volume the magnetic field is of such a strength that the magnetization of the magnetizable elements enters a saturated state when the magnetizable element is located in one of these subvolumes (or would enter a saturated state if the magnetizable element were to be located in one of these subvolumes). In the test volume the inhomogeneous magnetic field is however so low that the magnetization of the magnetizable elements does not yet enter a saturated state when the magnetizable element is located in the test volume. The range beneath the saturation point in respect of the magnetizable element is preferably nonlinear.

As the next step in the method according to the invention, a temporally variable magnetic field is then generated in the overall volume. In the event that the magnetizable element is located in a subvolume different from the test volume, the temporally variable magnetic field changes nothing to do with the fact that the magnetization of the magnetizable element is in a saturated state. There is therefore no change to the overall magnetization in the overall volume. The situation is different when the magnetizable element is located in the test volume: The variable magnetic field here causes a variation in the magnetization of the magnetizable element.

The magnetization in the overall volume can be measured by way of pick-up coils. The pick-up coils can also be identical to the generator coils. The following next step is thus possible according to the invention: A measurement signal generated by the magnetization in the overall volume in response to the temporally varying magnetic field is obtained. The measurement is evaluated. As mentioned above, the measurement signal is different according to where the magnetizable element is situated (in the test volume or not), with the result that information can be obtained as to whether the magnetizable element is situated in the test volume or not. As a rule, the magnetizable element will not be situated in the test volume immediately during the first pass. All the subvolumes will therefore be gone through in succession, in other words also selected as the test volume in each case. In other words, the steps involved in generating the temporally constant inhomogeneous magnetic field, the temporally variable magnetic field and in obtaining the measurement signal in response to the temporally varying magnetic field are repeated whilst varying the selection of the test volume from the subvolumes until such time as a subvolume is found in which the magnetizable element is situated. By preference, the steps are repeated until such time as all the subvolumes have been checked. Information about the subvolume in which the magnetizable element is situated is then made available. This information can be output as a data value by a computer control unit which deals with the execution of the method. The information can also be made available in the form of image information. In this situation, the subvolumes correspond to individual voxels (volume elements of a 3-D image). With regard to the simple version of the method described here, the information in the voxels is binary, in other words the magnetizable element is either located in the respective subvolume corresponding to the voxel, or it is not. The measurement signals obtained by means of the pick-up coil can however also be used in order to define a gray scale value for the voxel.

With regard to a preferred embodiment of the invention, the invasive instrument comprises a basic body made of non-magnetizable material, on (or in) which at least one magnetizable marker is fitted. The magnetizable element does not therefore need to be especially large. The marker suffices for localizing the invasive instrument. A small marker actually has the advantage that the subvolumes can be chosen to be small, thereby increasing the precision of the localization. The magnetizable marker can preferably comprise a magnetic fluid which is situated in a closed container on or in the basic body. Typically, for example, a closed hose on a catheter can contain a conventional contrast agent, as is used for example in core spin resonance (for example Resovist™ from the company Schering AG, Berlin).

With regard to a preferred embodiment, the magnetization of the magnetizable element beneath the saturation level changes nonlinearly with an external magnetic field. The temporally variable magnetic field can then exhibit a constant (basic) frequency. A signal generated by the magnetization is then picked up by the pick-up coil as a measurement signal, which is subjected to a Fourier transformation. The evaluation of the measurement comprises the capture and evaluation of the harmonics of the basic frequency in the measurement signal. The aforementioned harmonics occur as a result of the nonlinearity, even if the temporally variable magnetic field does not exhibit these harmonics on the input side. The presence of the harmonics can thus serve to determine whether or not the invasive instrument or its magnetizable element is situated in the respective test volume, whereby the nonlinearity particularly is utilized in the test volume. The signals obtained, for example the Fourier coefficients of the harmonics, can also be used directly for imaging purposes. The information made available at the end of the method according to the invention can be an image in which Fourier coefficients of the harmonics are assigned to the individual voxels. There can be a separate image for each harmonic. The individual components can however also be superimposed.

With regard to a further preferred embodiment, the coils which generate the inhomogeneous and the temporally variable magnetic field are situated in a fixed locational arrangement with respect to an x-ray system. By way of support, an x-ray image can be captured during the invasive intervention by the x-ray system (or a 3-D data record of x-ray images). A representation of the x-ray image (or of the 3D record) can then be made available in which the location of the magnetizable elements is marked. As a result of the fixed locational arrangement of the coils with respect to the x-ray system the respective location of the test volume is namely well-defined with respect to the x-ray system, with the result that the information obtained about the location of the invasive instrument is available directly in the same coordinate system in which the x-ray images are also captured. It thus becomes possible to simply “draw in” the invasive instrument in x-ray images.

The method according to the invention can be performed repeatedly in succession if the position (location) of the invasive instrument changes. A type of “tracking” preferably occurs here: In order to speed up the method, the subvolume, in which the magnetizable element was situated with regard to the previous position of the invasive instrument is initially selected as the test volume. The adjacent subvolumes are each subsequently defined as the test volume, then the subvolumes adjacent to these etc. Normally a subvolume in which the magnetizable element is situated will be found quite quickly if the difference between the two positions of the invasive instrument is not too great. By this means, time can clearly be saved in respect of the subsequent localization process in each case if a basic localization took place the first time the method was performed.

A new type of invasive instrument also forms part of the invention. Different from previous invasive instruments, in addition to a basic body it includes at least one magnetizable marker on the basic body.

By preference, the magnetization of the marker changes nonlinearly with the applied magnetic field. The magnetization curve of the marker should be chosen in total such that the method according to the invention is enabled. In particular, the magnetizable marker should enter a saturated state “early” in order that a saturated level of magnetization can be produced for the magnetizable marker through the provision of conventional coils without an excessive resource requirement.

This is realized particularly in the situation when the marker comprises a multiplicity of magnetizable small particles, such as is the case for example with a fluid contrast agent which is normally a (colloidal) suspension of magnetizable small particles. The fluid contrast agent must naturally be fitted somehow to the basic body. A suitable closed container is used for this purpose, which is added individually to the basic body or is situated inside the latter. This can be a hose, for example. A typical catheter consists for instance of plastic, which means that the hose can be manufactured at the same time as the catheter in the same process.

The invention also relates to a set of at least two invasive instruments according to the invention which either differ by virtue of the form or size of their marker or each have two markers, whereby they then differ by virtue of the spacing between the two markers. Particularly when the subvolumes are chosen to be sufficiently small and in the case of the aforementioned variant of the method according to the invention in which images are produced, it becomes possible to differentiate the two invasive instruments from one another on the basis of the information (or the images) provided by the method. A doctor can thus use two different invasive instruments and simultaneously still distinguish one from the other.

Preferred embodiments of the invention will be described in the following with reference to the drawings, in which;

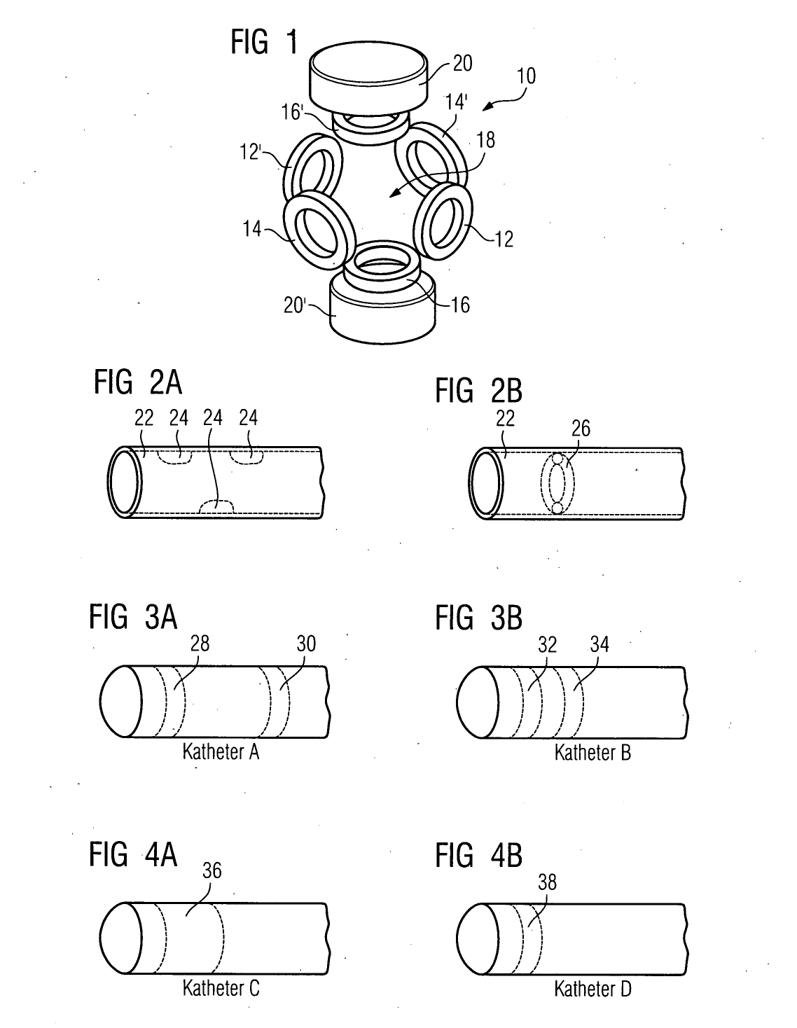

FIG. 1 gives a schematic illustration of the basic structure of the coil system which is used for the method according to the invention,

FIGS. 2A and 2B illustrate variants of catheters according to the invention,

FIGS. 3A and 3B illustrate catheters differing from one another on the basis of the spacings of markers, and

FIGS. 4A and 4B illustrate catheters with differing markers.

The invention uses the method disclosed in the aforementioned article by Gleich and Weizenecker in the present case for localizing an invasive instrument during an invasive intervention on a patient. The method described by Gleich and Weizenecker is thus used on a considerably wider scale for a new type of purpose.

The basic structure of a coil system, as is preferably used with regard to the present invention, is illustrated in FIG. 1:

In a coil system 10, three pairs of coils comprising the coils 12 and 12′, 14 and 14′, 16 and 16′ are provided which in the present case are arranged orthogonally with respect to one another. Almost any desired magnetic field can be set at the coil pairs by way of a power supply (not shown). In particular, with the aid of the coil system 10 it is possible to generate an inhomogeneous field in the inner area 18 in which the patient on whom the invasive intervention is being undertaken is situated. The inhomogeneous field generated here exhibits the following characteristic: The inhomogeneous field is defined as follows on the basis of the magnetization curve of one or more markers arranged on an invasive instrument, a catheter for example (see below): The magnetization curve of the marker should be non-linear and enter a saturated state relatively early with not an excessively high magnetic field applied. The inhomogeneous magnetic field now exhibits the characteristic whereby the magnetic field strength is so high across almost the entire inner area 18 that the magnetization actually enters a saturation state. Just in one small selected subvolume, preferably in the vicinity of a completely field-free point, the magnetic field should be so small that the magnetization curve of the marker does not enter a saturation state if the marker happens to be situated at this point.

The coil system 10 enables the aforementioned subvolume or the aforementioned field-free point to be arranged at almost any desired place in the inner area 18. The overall volume 18 can thus be divided into a large number of subvolumes, whereby each of the subvolumes in succession is the particular subvolume in which the magnetic field is low.

In order to perform the method the inhomogeneous magnetic field is then kept temporally constant during a test step while a sinusoidal magnetic field is generated by coils 20 and 20′.

The sinusoidal magnetic field has absolutely no effect in the case in which the marker on the invasive instrument is situated in a location at which the magnetization is made to enter a saturated state by the inhomogeneous constant magnetic field. It does however have an effect when the marker happens to be situated in the subvolume (test volume), in which the inhomogeneous magnetic field does not cause the marker to enter a saturated state. The magnetization of the marker should in particular behave non-linearly with the external magnetic field supplied by way of the coils 20 and 20′. As a result of the sine shape of the additional temporally varying magnetic field, a response curve for the magnetization which includes higher harmonics than the sine frequency used results by virtue of the nonlinearity of the magnetization curve of the marker. Such a response signal can for example likewise be picked up with the aid of the coils 20 and 20′. The coils 20 and 20′ can thus act both as generator coils and also as pick-up coils.

The measurement signal picked up can then be evaluated, for example by means of a Fourier transformation. The Fourier coefficients of the higher harmonics are only other than zero when the marker is situated in the test volume, in other words if its magnetization has not yet entered a saturation state. A unified image of the entire interior 18 of the coil system 10 results from the passage of the overall volume 18, in other words from shifting the location of the test volume.

The invasive instrument reveals itself through its marker, and its location thus becomes known. The desired localization has thus taken place. For further details of the method, reference should be made to the article by Gleich and Weizenecker.

Alternatives are shown in FIGS. 2A and 2B as to how an invasive instrument which is used with regard to the method according to the invention could look. The tip of a catheter 22 is shown in FIGS. 2A and 2B. The tip is represented as a tube, as is the case with typical catheters.

A plurality of plastic pockets 24 are situated in the interior of the tube, in accordance with FIG. 2A. Each plastic pocket 24 contains a conventional magnetic contrast agent, for example Resovist™ from the company Schering AG, Berlin.

In the case of the alternative according to FIG. 2B, a single continuous hose 26 is situated inside the tube. This hose 26 also contains a magnetic contrast agent.

Magnetic contrast agents are normally magnetic fluids. Magnetic fluids are colloidal suspensions containing small magnetizable particles.

The magnetic particles contained in the pockets 24 or in the hose 26 have the magnetization curve suitable for the method according to the invention. The magnetization can be measured by the coils 20 and 20′(FIG. 1), in particular the response of the magnetization to a temporally varying magnetic field delivered through the coils 20 and 20′ while an inhomogeneous magnetic field is simultaneously provided through the coils 12, 12′, 14, 14′, 16, 16′.

With appropriately careful control of the coil system 10 and a computer evaluation of the aforementioned Fourier coefficients in the response signals which are picked up by the coils 20 and 20′, it is possible to generate a three-dimensional image in which the pockets 24 or the hose 26 are mapped. The attending doctor can thus recognize the catheter tip 22 in the image.

A plurality of catheters are used for some invasive interventions. In order to distinguish these catheters from one another, provision can be made according to FIG. 3A for one catheter to have two markers 28 and 30 (after the fashion of the pockets 24 or the hose 26) spaced relatively far from one another, while in a catheter B illustrated in FIG. 3B markers 32 and 34 are arranged relatively close to one another. The method according to the invention permits a sufficiently precise data capture in order to ensure that catheter A and catheter B can be distinguished from one another. This can be done by the doctor, for example, by viewing a corresponding image.

Two catheters C and D can also differ by virtue of the size of their marker. For example, according to FIG. 4A one catheter C is equipped with a relatively large marker 36 (for example after the fashion of a broadened hose 26), while catheter D is provided with a relatively narrow (tube-shaped, for example) marker 38. Here too the method according to the invention is sufficiently refined in order to enable differentiation between catheters C and D.

The invention thus makes it possible to localize certain invasive instruments, namely such as are shown for example in FIGS. 2A and 2B, with the aid of simple magnetic fields (coil system 10). A magnetizable marker simply needs to be provided on the invasive instruments (catheters 22 or catheter A to catheter D), for example on the tip. When compared with the position sensors according to the prior art, this represents a relatively straightforward means of equipping the invasive instruments. No electrical connection is required from the tip of the invasive instrument to the other end of the instrument.

1.-12. (canceled)

13. A method for localizing an invasive instrument during an invasive intervention on a patient, comprising:
arranging a magnetizable element in the invasive instrument;
dividing an overall volume comprising a path of the invasive instrument from an entry point into the patient to a target point of the intervention into a plurality of subvolumes;
selecting one of the subvolumes as a test volume;
generating an inhomogeneous magnetic field that is temporally constant for a duration of a test step in the overall volume;
setting up the inhomogeneous magnetic field so that a magnetization of the magnetizable element:
enters a saturated state in all other subvolumes except for the test volume if the magnetizable element is located in one of the all other subvolumes, and
does not enter a saturated state if the magnetizable element is located in the test volume;
creating a temporally variable magnetic field in the overall volume;
obtaining a measurement signal generated by a magnetization of the overall volume in response to the temporally variable magnetic field;
evaluating the measurement signal to decide whether the magnetizable element is located in the test volume; and
locating the invasive instrument based on the evaluation.

14. The method as claimed in claim 13, wherein the steps of selecting, generating an inhomogeneous magnetic field, setting up the inhomogeneous magnetic field, creating a temporally variable magnetic field, obtaining a measurement signal, and evaluating the measurement signal are repeated until a subvolume where the magnetizable element is located has been found.

15. The method as claimed in claim 14, wherein the subvolume where the magnetizable element is located is selected as an initial test volume for a successive position of the invasive instrument moving along the path and adjacent subvolumes are selected successively as the test volume until a further subvolume where the magnetizable element is located has been found.

16. The method as claimed in claim 13, wherein the invasive instrument comprises a non-magnetizable basic body and the magnetizable element is arranged on or in the basic body.

17. The method as claimed in claim 16,
wherein the magnetizable element is a magnetizable marker,
wherein the magnetizable marker comprises a magnetic fluid contained in a closed container, and
wherein the closed container is arranged on or in the basic body.

18. The method as claimed in claim 13, wherein the magnetization of the magnetizable element under a saturation level changes non-linearly with the temporally variable magnetic field.

19. The method as claimed in claim 13, wherein the temporally variable magnetic field comprises a constant basic frequency and a harmonics of the basic frequency in the measurement signal is captured and evaluated.

20. The method as claimed in claim 13, wherein the inhomogeneous magnetic field and the temporally variable magnetic field are generated by coils located in a fixed location arrangement with respect to an x-ray system.

21. The method as claimed in claim 13, wherein the x-ray system captures an x-ray image of the patient and a location of the magnetizable element is marked in the x-ray image.

22. An invasive instrument used in a medical procedure, comprising:
a non-magnetizable basic body; and
a magnetizable marker arranged on the basic body.

23. The invasive instrument as claimed in claim 22, wherein a magnetization of the magnetizable marker changes non-linearly in a magnetic field.

24. The invasive instrument as claimed in claim 22, wherein the magnetizable marker comprises a fluid contrast agent contained a closed container arranged on or in the basic body.

25. The invasive instrument as claimed in claim 24, wherein the fluid contrast agent comprises a plurality of magnetizable small particles.

26. The invasive instrument as claimed in claim 22, wherein a plurality of invasive instruments are used in the medical procedure.

27. The invasive instrument as claimed in claim 26, wherein each of the invasive instruments comprises a different size of a magnetizable marker.

28. The invasive instrument as claimed in claim 26, wherein each of the invasive instruments comprises two magnetizable markers with a different spacing between the two markers.

29. A medical system for performing an invasive intervention on a patient, comprising:
an invasive instrument comprising a magnetizable element that inserts into the patient in an overall volume from an entry point into to a target point of the intervention;
a first magnetic field generator that generates an temporally constant inhomogeneous magnetic field in the overall volume for a duration of a test step;
a second magnetic field generator that generates a temporally variable magnetic field in the overall volume; and
an evaluation device that evaluates a magnetization of the overall volume in response to the temporally variable magnetic field and locates the invasive instrument based on the evaluation.

30. The medical system as claimed in claim 29, wherein the temporally variable magnetic field comprises a constant basic frequency and a harmonics of the basic frequency in the measurement signal is captured and evaluated.

31. The medical system as claimed in claim 29,
wherein the overall volume is divided into a plurality of subvolumes and one of the subvolumes is selected as a test volume,
wherein the inhomogeneous magnetic field is generated so that a magnetization of the magnetizable element:
enters a saturated state in all other subvolumes except for the test volume if the magnetizable element is located in one of the all other subvolumes, and
does not enter a saturated state if the magnetizable element is located in the test volume,
wherein the magnetization of the overall volume is evaluated to decide whether the magnetizable element is located in the test volume, and
wherein a further subvolume is repeatedly selected as the test volume and the inhomogeneous magnetic field is repeatedly generated and the magnetization of the overall volume is repeatedly evaluated until a subvolume where the magnetizable element is located has been found.

32. The medical system as claimed in claim 31, wherein the subvolume where the magnetizable element is located is selected as an initial test volume for a successive position of the invasive instrument moving along a path and adjacent subvolumes are selected successively as the test volume until a subvolume where the magnetizable element is located at the successive position has been found.
